(19) United States
(12) Patent Application Publication
(10) Pub. No.: US 2009/0263842 A1
(43) Pub. Date: Oct. 22, 2009

(54) METHOD OF ASSESSING THE METASTATIC STATUS OF A PRIMARY TUMOR

(75) Inventor: David B. Krizman, Gaithersburg, MD (US)

Correspondence Address: PROSKAUER ROSE LLP, 1001 PENNSYLVANIA AVE, N.W.,, SUITE 400 SOUTH, WASHINGTON, DC, 20004, US

(73) Assignee: EXPRESSION PATHOLOGY, Gaithersburg, MD (US)

(21) Appl. No.: 12/294,423

(22) PCT Filed: Mar. 26, 2007
(86) PCT No.: PCT/US2007/064924

§ 371 Date: Mar. 27, 2009

Related U.S. Application Data
(60) Provisional application No. 60/785,364, filed on Mar. 24, 2006.

(51) Int. Cl. C12Q 1/34; C12Q 1/02

(52) U.S. Cl. 435/18; 435/29

(57) ABSTRACT

The current invention provides a method for determining the metastatic potential, capability, status, or characteristics of a primary tumor from a human cancer patient by determining expression patterns of proteins that initiate, cause, promote, mediate, inflict, or otherwise aid metastatic properties of cells from a primary tumor. The identification of one or more proteins associated with metastasis in a single primary tumor can determine whether a primary tumor is metastatic or has the potential to become metastatic.

### PRIORITY DATA AND INCORPORATION BY REFERENCE

This application claims the benefit of priority of U.S. Provisional Patent Application No. 60/785,364, filed Mar. 24, 2006, which is incorporated herein by reference in its entirety.

### FIELD OF THE INVENTION

The present invention provides a method of assessing the metastatic status of a primary tumor or the potential of a primary tumor to progress to metastatic disease by analyzing the proteome of a sample derived from the primary tumor. Determination of the potential for metastasis of a primary tumor has direct impact on the stage of the cancer, the aggressive behavior of the cancer, the course of therapeutic action, and patient prognosis.

### BACKGROUND OF THE INVENTION

Trained pathologists and histotechnologists perform molecular analyses on cancer tissue to determine the expression of specific molecular biomarkers. Information derived from such analyses provide valuable prognostic tools. For instance, a number of different protein biomarkers are associated with specific stages or grades of cancer and are relied upon to guide therapies. However, current methods, such as immunohistochemistry (IHC), generally are capable of analyzing only a single biomarker at one time. Thus, if a single protein biomarker is highly predictive of a specific stage or grade of cancers, an immunohistochemistry approach can be very effective. If many biomarkers need to be analyzed simultaneously, however, the IHC approach is too labor intensive, technically difficult, and cost prohibitive.

One of the most important factors to be understood by a clinician in order to provide for optimal treatment is the metastatic status or potential of a primary tumor. This is because it is the metastasis, and not the primary tumor that usually leads to the demise of the cancer patient. Metastasis is the process by which cancerous cells from a primary tumor acquire, through genetic or proteomic changes, the ability to dissolve the surrounding tissue that contains them and releases them to travel to and invade surrounding tissue or enter the blood system and travel to distant sites. Currently, there are no molecular methods that can reliably predict or indicate whether or not a primary tumor has already metastasized or likely will metastasize. Many individual protein biomarkers have been shown to be involved in the metastatic process, but a single protein biomarker has not proven predictive of metastasis and thus no current molecular analysis is capable of providing such important information. Thus new molecular methods and information to indicate the metastatic status or potential of a primary tumor are highly desirable.

This invention is directed to a novel approach to determine the metastatic status or predict the potential for a primary tumor to become metastatic by determining the expression of protein biomarkers. Many individual proteins have previously been shown to be involved in the metastasis of primary tumors, and expression of these multiple proteins cannot be assessed simultaneously using the current methodology of IHC. Thus, a need exists for the present invention, in which one or more proteins are analyzed in concert, and in the context of this invention, will indicate whether a primary tumor is already metastatic or will likely become metastatic.

### SUMMARY OF THE INVENTION

In one aspect, the invention provides a method of assessing a metastatic status of a test primary tumor by identifying one or more test proteins expressed by the test primary tumor to create a test protein profile, and comparing the test protein profile with a reference protein profile that contains one or more reference proteins and is obtained from modified samples of one or more reference primary tumors that are known to have become metastatic, where the presence of a protein in the test protein profile and the reference protein profile indicates the metastatic status of the test primary tumor. In embodiments, the modified samples of one or more primary tumors contain histopathologically processed fresh tissue or frozen tissue. The histopathologically processed fresh tissue or frozen tissue is contacted with a reaction buffer and heated at a temperature and for a time sufficient to reduce protein cross-linking in the tissue, and then treated with an effective amount of a proteolytic enzyme for a time sufficient to disrupt tissue and cellular structure of the tissue. The histopathologically processed frozen or fresh tissue is formalin-fixed tissue, formalin-fixed cells, formalin-fixed and paraffin embedded (FFPE) tissue, FFPE cells, FFPE tissue blocks, FFPE cells from primary tumors, FFPE tissue blocks from biopsies obtained surgically from primary tumors, or formalin fixed or paraffin embedded tissue culture cells derived from a primary tumor. In certain embodiments, the test protein profile and the reference protein profile each contain two or more proteins. For example, the reference protein profile contains one or more proteins (e.g., one, two, three, four, five, ten, fifteen, twenty, thirty, fifty or one hundred or more) listed in Tables 1-4. In some embodiments, the modified samples contain protein samples prepared using Liquid Tissue® reagents and protocols. The reference protein profile is obtained from modified reference samples of two or more primary tumors. In some embodiments, the test primary tumor is of the same type of tumor as the reference primary tumor. The test primary tumor is, e.g., an ovarian, kidney, lung or breast tumor.

The reference protein profile contains a matrix metalloprotease, a metalloprotease, a cadherin, a protocadherin, an integrin, an extracellular matrix protein, an enzyme that digests extracellular matrix, a motility protein, a cell adhesion protein, a membrane invasion protein, a membrane disintegration protein, a basement membrane disintegration protein, a basement membrane invasion protein, or a cell motility protein. The one or more test proteins are identified by mass spectroscopy. Further, the one or more reference proteins are identified by a mass spectroscopy technique. Mass spectrometry techniques include LC-ESI-MS, LC-ESI-MS/MS, LC-nanospray-MS, LC-nanospray-MS/MS, MALDI-TOF, MALDI-TOF/TOF, SELDI-TOF, and SELDI-TOF/TOF. The identification of one or more test proteins is performed using a protein array or an immuno-based assay.

In another aspect, the invention provides a method of assessing a metastatic status of a test primary tumor by determining the amount one or more test proteins expressed by the test primary tumor to create a scaled test protein profile, and comparing the test protein profile with a reference protein profile, wherein the reference protein profile contains the relative amounts of one or more reference proteins and is obtained from modified samples of one or more reference primary tumors that are known to have become metastatic. The presence of a protein in the scaled test protein profile at a level about equal to or greater than the amount of the protein in the reference protein profile indicates the metastatic status of the test primary tumor.

In a further aspect, the invention provides a method for generating a metastatic tumor protein profile by collecting a first sample containing material obtained from a first primary tumor that is known to have metastasized in a subject, where the material contains at least one protein expressed in a tumor cell of the first primary tumor and collecting a second sample containing material obtained from a second primary tumor that is known to have not metastasized in a subject, where the material contains at least one protein expressed in a tumor cell of the second primary tumor. The method also includes the step of separately modifying the first sample and the second sample by contacting the material with a reaction buffer and heating the contacted material at a temperature and for a time sufficient to negatively affect protein cross-linking in the tissue, and treating the heated material with an effective amount of a proteolytic enzyme for a time sufficient to disrupt the tissue and cellular structure of the tissue. The method also includes the steps of determining the amount one or more proteins in the modified first and second samples and identifying proteins that are detectable in the first sample but not the second sample, and detectable in higher amounts in the first sample than in the second sample, thereby generating a metastatic tumor protein profile.

In yet another aspect, the invention provides a kit containing in one or more containers two or more proteins (e.g., one, two, three, four, five, ten, fifteen, twenty, thirty, fifty or one hundred or more), or antibodies that are immunospecific therefor, as listed in Tables 1-4.

Other objects, features, and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWING

FIG. 1 is a flow chart that illustrates one embodiment of the invention for analyzing proteins and peptides from soluble lysates from primary tumors by mass spectrometry.

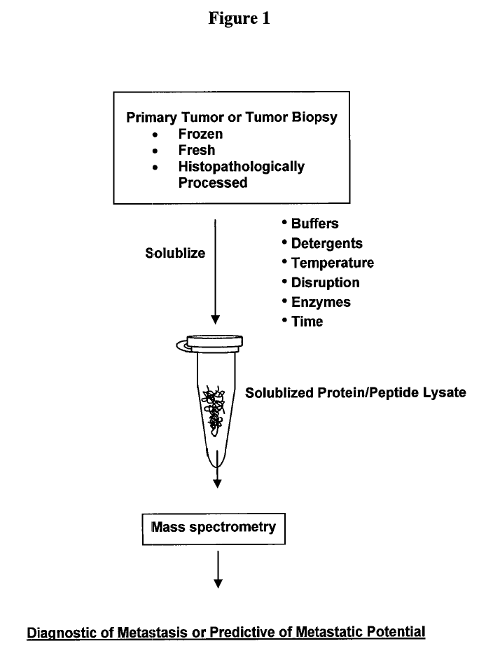

### DETAILED DESCRIPTION OF THE INVENTION

In one embodiment, the invention is directed to a method of assessing the status potential of a primary tumor to metastasize, the method comprising the steps of identifying proteins expressed in a protein-containing sample obtained from a primary tumor to create a protein profile for said primary tumor and then comparing the protein profile of the primary tumor with a reference protein profile from primary tumors that are known to have become metastatic. When the proteins in the reference protein profile are present in the protein profile from the primary tumor, the primary tumor has the potential to become metastatic.

“Primary tumor” is the nomenclature used when a tumor has originated in the same organ, and has not metastasized to it. “Metastasis” is the transfer of a disease from one organ or part to another not directly connected with it. “Proteins” are long and short amino acid chains that may be branched or unbranched. “Proteomics” refers to the large scale study of proteins. For instance, all proteomic technologies rely on the ability to separate a complex mixture so that individual proteins are more easily processed with other techniques. Proteomics also involve the identification of specific proteins. For instance, identification may involve the low-throughput sequencing through Edman degradation. Higher-throughput proteomic techniques are based on mass spectrometry, commonly peptide mass fingerprinting on simpler instruments, or de novo repeat detection sequencing on instruments capable of more than one round of mass spectrometry. Antibody-based assays can also be used, but are unique to one sequence motif. Other fields of proteomics involve the quantification of protein, the sequence analysis of proteins, the structure of proteins, the interaction of proteins, the modification of proteins and the mapping of the location of proteins in the cell.

Indeed, the recent completion of the Human Genome Project, has spurred dramatic advances in the field of genomics and proteomics. “Functional genomics” attempts to determine the physiological role of each gene. An important step in discovering the function of each gene is to carefully measure the expression patterns of mRNA transcripts in tissue specimens. By measuring specific expression patterns of genes and gene products such as mRNA, one can determine what genes are expressed and at what levels in a normal, healthy cell type. Moreover, by measuring the expression patterns in diseased cell types, new insight can be gleaned into the pathological progression of that disease. In addition, new markers may be discovered, thereby yielding new diagnostic and therapeutic strategies.

However, despite the power of understanding gene expression patterns in disease processes, there is not always direct correlation between RNA transcription of a gene and the expression pattern of the protein derived from that gene. It is primarily the expression of proteins that dictate cell function and phenotype. Thus, it is the specific analysis of the protein content of cells from diseased and normal tissue, and not gene transcription analysis, that directly determines the phenotypic and/or clinical characteristics of the cells derived from that tissue. The present invention strives to determine specific expression patterns of entire groups of proteins simultaneously so as to understand the cellular phenotype of metastasis. The information derived from this analysis will provide direct correlations between protein biomarkers and tissue pathology and thereby enable improved diagnostic and prognostic evaluations.

Thus the present invention is involved with the identification of proteins in tumor samples. The invention specifically utilizes protein expression analysis to determine “protein profiles,” i.e. the concerted expression of proteins known to initiate, cause, promote, mediate, inflict, or otherwise aid metastatic properties of cells within a primary tumor such that when large numbers of these proteins, groups of proteins, collections of proteins, or protein families, for example 5-10 proteins from one or more different groups and/or families of proteins instead of one, are identified as expressed in a primary tumor are indicative of the positive metastatic phenotype or the potential for displaying metastatic properties of cells within the primary tumor.

“Reference protein profiles” are the collection of proteins that have been shown to initiate, cause, promote, mediate, inflict or otherwise aid metastatic properties of cells within a primary tumor and are indicative of imparting metastatic properties upon a primary tumor. Such reference protein profiles can include but are not limited to the following proteins: matrix metalloproteases, cadherins, protocadherins, integrins, extracellular matrix proteins, enzymes that digest extracellular matrices, motility proteins, cell adhesion proteins, or cell motility/mobility proteins. A reference protein profile is a reference point and is developed by identifying a change of expression of large numbers of these proteins in concert, not just one, or groups of these proteins, or families of proteins, and not just one protein from a specific group of proteins or protein collections, that is indicative of the metastatic properties or potential metastatic properties of cells within primary tumors. It should be noted that not all of these proteins in a reference protein profile need be present or identified in a single sample from a primary tumor to indicate the metastatic potential or metastatic characteristics or status of the primary tumor. However, there does need to be identification of many proteins from at least one or more of the types of proteins described in the reference protein profile. The greater the number of these proteins that are found to be expressed, the greater the chance of positive diagnosis of metastasis or the greater likelihood that a primary tumor is already metastatic or will become metastatic.

A reference protein profile may be created by analyzing a sample from more than one primary tumor. In one embodiment, the reference protein profile is created by analyzing a sample from primary tumors that are the same type of primary tumor being assessed to determine its metastatic status or potential.

A scaled test protein profile is created by determining the presence and relative amounts of one or more test proteins expressed by the primary tumor. The relative amount of a test protein is determined using methods known in the art. For example, the amount of a protein in a sample is measured against total protein in the sample, or alternatively, is measured relative to an internal standard protein that does not vary significantly between multiple samples. In certain embodiments, a reference protein profile contains information regarding the relative amounts of one or more reference proteins in one or more reference primary tumors that are known to have become metastatic.

Specifically, the present inventors have found that the expression of the variety of metastasis-associated proteins i.e., the proteins of the reference protein profile, are upregulated in their expression profile in cells derived from primary tumors that had metastasized but not in cells from primary tumors that had not metastasized. It is the expression of these proteins that correlate with the likelihood that a primary tumor will metastasize or has already metastasized beyond the primary tumor site. It is the fact that primary tumors metastasize, or gain the ability to invade surrounding tissue or distant tissue within the same body, that eventually leads to death of the patient. If tumors did not metastasize then most cancers could be simply cured by surgical removal of the primary tumor. However, most primary tumors, if left untreated, eventually acquire the ability to invade and multiply in vast numbers across many regions of the body. This fact eventually leads to the demise of the patient. Thus, it is of value to know if a primary tumor has metastasized, or has already acquired the capability to metastasize. This leads to improved ability of clinical professionals to treat the cancer patient.

In particular embodiments, that present invention employs a method of assessing a metastatic status of a test primary tumor (e.g., whether the primary tumor has become metastatic or has an increase potential to become metastatic) by comparing a test profile of proteins identified in the primary tumor of interest (the “test primary tumor”) with a reference profile generated from primary tumors known to have become metastatic. Such reference profiles are thus useful as diagnostic tools in cancer, such as kidney, breast, lung, or ovarian cancer. Thus, in certain embodiments, the present invention concerns novel compositions comprising at least one protein, and kits containing one or more proteins, or antibodies that are immunospecific for the proteins. As used herein, a “protein” generally refers, but is not limited to, a peptide or polypeptide of greater than about 2 amino acids or the full length endogenous sequence translated from a gene. “Protein” and “polypeptide” are terms that have the meaning described herein and may be used interchangeably herein.

In certain embodiments the size of a test or reference protein may contain, but is not limited to, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 110, about 1120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 1000, about 1200, about 1500 or greater amino acid residues, and any range derivable therein.

The present invention is not limited to the particular methodologies, protocols, constructs, formulae and reagents described but further include those known to the skilled artisan. It is also to be understood that the terminology used herein is for the purpose of describing particular embodiments only, and is not intended to limit the scope of the present invention.

As used herein and in the appended claims, the singular forms “a,” “and,” and “the” include plural reference unless the context clearly dictates otherwise. Thus, for example, reference to “a cell” is a reference to one or more cells and includes equivalents thereof known to those skilled in the art, and so forth.

Unless defined otherwise, all technical and scientific terms used herein have the same meaning as commonly understood to one of ordinary skill in the art to which this invention belongs. Any methods, devices, and materials similar or equivalent to those described herein can be used in the practice or testing of the invention. All publications and patents mentioned herein are incorporated herein by reference.

### Biological Samples

According to one embodiment, the “sample” in the above described method may be a biopsy obtained from a primary tumor. For instance, samples may be tissue obtained from biopsies, diagnostic surgical pathology or cytology. Any biological sample that has been removed from an organism that has a primary tumor is a sample according to the present invention. Examples of methods of actually “obtaining” a biological sample include but are not limited to using a manual core punch, tissue punch, laser-based tissue microdissection and other techniques that are well known in the arts. The actual size of the obtained biological sample is not important as long as there is a sufficient amount to perform the chosen assay.

It is understood that a primary tumor tissue and/or biopsy obtained from a primary tumor can take the form of frozen tissue, fresh frozen, histopathologically processed (chemical fixation such as formalin fixation), or fresh (not fixed or frozen).

A modified sample includes a biological sample that is physically and/or chemically manipulated to increase detectability of a protein or proteins contained within the sample.

One embodiment of the present invention provides for obtaining a soluble protein/peptide lysate from a histopathologically processed primary tumor or a histopathologically processed biopsy obtained from a primary tumor. Examples of histopathological processing of biological samples include, but are not limited, to: formalin fixation of tissues or cells; formalin fixation/paraffin embedding of tissues or cells; and formalin fixation and/or paraffin embedding of tissue culture cells.

Histopathological processing typically occurs through the use of a formalin fixative. Formalin is used widely because it is relatively inexpensive, easy to handle and, once the formalin-fixed sample is embedded in paraffin, the sample is stored easily. Additionally, formalin is often the fixative of choice because it preserves both tissue structure and cellular morphology. Although the exact mechanism may not be understood fully, fixation occurs by formalin-induced cross-linking of the proteins within the biological specimen. Due to these protein cross-links, formalin fixation has found wide success in the traditional microscopic analysis of histologic sections. A novel set of reagents and protocol (Liquid Tissue® U.S. patent application Ser. No. 10/796,288, filed Mar. 10, 2004 and published as US patent application publication number 20050014203, which is incorporated herein by reference in its entirety) was developed that solubilizes protein and peptides directly from formalin fixed tissue for analysis in biochemical assays. Prior to the Liquid Tissue® technology, only a few experimental techniques were available for analysis of proteins and peptides from histopathologically processed tissue samples, including histopathologically processed primary tumors and histopathologically processed biopsies obtained from primary tumors, due to the insolubility of the protein as a result of the formalin fixation, and there are deficiencies in each of these experimental techniques. Prior to the Liquid Tissue® technology, formalin fixation rendered formalin fixed pathologic tissue, including histopathologically processed primary tumors and histopathologically processed biopsies obtained from primary tumors, of little value for many of the powerful analysis methods that have been developed in recent years, such as mass spectrometry and protein arrays.

Formalin fixation for processing and storage of surgically removed tissue and biopsies, including those tissues and biopsies derived from primary tumors, is universal. For nearly the last one hundred years, body tissue has been routinely fixed in formalin or formalin fixed/paraffin wax-embedded (FFPE) blocks. The overwhelming majority of hospitals and pathology laboratories, in the course of diagnostic surgical and anatomic pathology, process all tissue with formalin prior to use for many diagnostic tests.

In one embodiment, the invention provides for the creation of a soluble protein/peptide lysate from histopathologically processed primary tumors and histopathologically processed biopsies of primary tumors. This method includes making a soluble protein/peptide lysate directly from a histopathologically processed primary tumor or biopsy, e.g., a tissue or cellular sample, allowing one to obtain, extract, isolate, solubilize, fractionate, and store substantially all of the protein/peptides contained within the sample. This soluble protein/peptide lysate forms a representative library of all of the proteins/peptides as they existed within the histopathologically processed primary tumor or histopathologically processed biopsy obtained from a primary tumor at the time of sampling.

If the sample is embedded in paraffin or some similar material, the paraffin may be removed by, for example, adding an organic solvent, heating; heating and adding a buffer comprising Tris, and/or heating and adding an organic solvent. Advantageously this step is carried out prior to the main heating step. If the sample is heated as part of the process to remove paraffin, the heating need only be brief, for example a few minutes. This brief heating advantageously may be repeated two or more times to ensure maximum removal of paraffin.

At any stage, the sample may be mechanically disrupted by, for example manual homogenization; vortexing; and/or physical mixing. The lysate produced by these methods may be subjected to a wide variety of biochemical assays. The lysate also may be fractionated, for example into nucleic acid and protein fractions, before assay. Each biomolecule fraction typically contains distinct and separate biomolecules that are suitable for use in biochemical assays.

The heating step may be carried out, for example, at a temperature between about 80° C. and about 100° C. and for a period of from about 10 minutes to about 4 hours. The proteolytic enzyme treatment lasts, for example, for a period of time from about 30 minutes to about 24 hours. The proteolytic enzyme treatment may be carried out, for example, at a temperature between about 37° C. to about 65° C. In each step, the reaction buffer may comprise a detergent, and/or a detergent may be added after the protease treatment. The detergent may be, for example, Nonidet P40, SDS, Tween-20, Triton X, and/or sodium deoxycholate, although the skilled artisan will recognize that other detergents may be used. The proteolytic enzyme may be for example, proteinase K, chymotrypsin, papain, pepsin, trypsin, pronase, and/or endoproteinase Lys-C, although the skilled artisan will recognize that other enzymes may be used. The reaction buffer may comprise Tris and may have a pH in the range of about 6.0 to about 9.0.

Furthermore, this protein/peptide lysate is capable of being serially diluted. Another embodiment of the present invention provides for a method wherein a protein/peptide lysate from a histopathologically processed primary tumor or histopathologically processed biopsy obtained from a primary tumor may be used in a number of experimental biochemical assay techniques including but not limited to mass spectrometry, western blotting, immuno-based assays, and protein arrays, to achieve the goal of the invention.

### Analysis of Biological Sample

The sample according to the present invention may be processed by the Liquid Tissue® technology wherein the soluble lysate is capable of being used with numerous experimental and diagnostic techniques.

### Method of Analysis

The present invention involves biochemical analysis of test protein (or “analyte”) within cells from a primary tumor or biopsy obtained from a primary tumor either by removing the cells from the tissue and preparing a soluble protein/peptide lysate or by analyzing analytes as they reside in intact tissue in a thin section. As used herein, the term “analyte” refers to a specific protein or peptide derived from a specific protein contained within a soluble protein/peptide lysate that is detectable by the technology of mass spectrometry or protein arrays.

The soluble lysate, and fractions thereof, are assayed by methodologies that include but are not limited to mass spectrometry, western blotting, immuno-based assays, and protein arrays for determination of the increased and/or differential expression of entire groups or multiple members of various groups of metastasis-associated proteins in primary tumors that have metastatic characteristics vs. primary tumors that do not have metastatic characteristics. Examples of mass spectrometry instrument formats include but are not limited to LC-ESI-MS, LC-ESI-MS/MS, LC-nanospray-MS, LC-nanospray-MS/MS, MALDI-TOF, MALDI-TOF/TOF, SELDI-TOF, and SELDI-TOF/TOF. Examples of metastasis-associated proteins are: matrix metalloproteases, metalloproteases, cadherins, protocadherins, integrins, extracellular matrix proteins, enzymes that digest extracellular matrices, motility proteins, cell adhesion proteins, membrane invasion proteins, membrane disintegration proteins, basement membrane disintegration proteins, basement membrane invasion proteins or cell motility/mobility proteins. It is understood that many types of proteins in the archive protein profile associated with the metastatic phenotype and metastatic characteristics of cells in primary and metastatic tumors. As such, the above protein examples are not intended to form a limited list and one skilled in the art will understand that other proteins not listed above may be part of the archive protein profile.

In the present invention, each lysate of soluble protein/peptides forms a representative library of specific proteins that directly reflects the status of those proteins in the primary tumor and/or tumor biopsy obtained from the primary tumor. An example of such a representative biomolecular lysate library from a histopathologically processed primary tumor and/or histopathologically processed tumor biopsy obtained from the primary tumor would be the preparation of a lysate of proteins and peptides directly from formalin-fixed paraffin embedded tissue/cells. The resulting preparation of biomolecules is characteristic of those expressed proteins that resided in the histopathologically processed biological sample, and that when assayed by mass spectrometry, do directly reflect the expression pattern or protein profile of the primary tumor. Thus, in accordance with the invention, the proteins detected in such primary tumor sample comprises a protein profile that is then compared with the proteins in the archive protein profile.

When the biomolecule of interest is a peptide, the peptide is directly derived from a previously intact protein from the extract in a soluble liquid form and the peptide and/or collection of peptides derive from and is representative of the total protein content of the cells procured from the starting primary tumor and/or tumor biopsy obtained from the primary tumor. As with proteins, any resulting peptide extract from the starting protein extract can be placed in any number of peptide/protein identification, analysis, and expression assay assays including but not limited to mass spectrometry, western blots, immuno-based assays, and protein arrays.

### Array Assays

A specific example of a high-throughput assay is the protein array. Protein arrays are highly parallel (multiplexed) and can be conducted in miniature (microarray). Protein arrays are quick, usually automated, highly sensitive, and capable of generating an enormous amount of data in a single experiment. The protein array is essentially a miniaturized version of the familiar ELISA or dot blotting immunoassay. Similar to ELISA and dot blots, protein array results are usually obtained using optical detection methods, for example fluorescent detection. The data generated by a single protein array experiment often is so voluminous that specialized computer software may be required to read and analyze the data that is generated.

### Description of the Tables

Tables 1-4 list specific proteins identified by mass spectrometric analysis of modified samples of multiple primary tumors. Fifteen tissue samples were obtained from primary tumors whose metastatic status was known (eleven samples were from tumors known to have metastasized, and four samples were from non-metastatic tumors) and modified as described herein. The modified samples were subjected to tandem mass spectrometry and all detected peptides were identified using commercial software (SEQUEST®, Thermo Fisher Scientific Inc.). Protein data were analyzed using Microsoft Access® to develop two large protein expression datasets representing metastatic or non-metastatic tumor samples. One large dataset is those proteins identified in one or more of the eleven metastatic primary tumors and the other large dataset is those proteins that are identified in one or more of the non-metastatic primary tumors. The protein differences and commonalities between these datasets were then ascertained.

Table 1 shows a list of proteins identified by mass spectrometry analysis of soluble protein lysates derived from a collection of histopathologically processed primary tumors originating from four different tissue types where the tumors are non-metastatic. No proteins that are known to initiate, cause, promote, mediate, inflict, or otherwise aid metastatic properties of cells from a primary tumor were identified.

Table 2 is a list of proteins identified by mass spectrometry analysis of soluble protein lysates derived from a collection of histopathologically processed primary tumors originating from five different tissue types where the primary tumors have metastatic characteristics and that had metastasized to either the surrounding tissue or localized lymph nodes. Note the identification of collections and concerted collections of multiple proteins in concert and multiple families of proteins that initiate, cause, promote, mediate, inflict, or otherwise aid metastatic properties of cells within a primary tumor.

Tables 3A-F show results from protein expression analysis by mass spectrometry of multiple proteins and/or multiple families of proteins that are know to initiate, cause, promote, mediate, inflict, or otherwise aid metastatic properties of cells within a primary tumor across multiple different primary tumor samples from multiple tissue types. The types of cancers are kidney, breast, lung, and ovarian cancer. The single common characteristic among all of these samples is that they are all primary tumors that are metastatic (having given rise to invasive metastases) and are found to express collections and/or concerted collections of multiple proteins and multiple families of proteins that are known to initiate, cause, promote, mediate, inflict, or otherwise aid metastatic properties of cells within a primary tumor.

Tables 4A-D show results from protein expression analysis of primary tumors, as determined by mass spectrometry, where the identified proteins do not come from the group of multiple proteins and/or multiple families of proteins that are known to initiate, cause, promote, mediate, inflict, or otherwise aid metastatic properties of cells within a primary tumor across multiple different primary tumor samples from multiple tissue types. The types of cancers are kidney, breast, lung, and ovarian cancer. The single common characteristic among these samples is that they are primary tumors that have not shown the metastatic phenotype or characteristics and have been determined not to express collections and/or concerted collections of multiple proteins and multiple families of proteins that are know to initiate, cause, promote, mediate, inflict, or otherwise aid metastatic properties of cells within a primary tumor. These samples are correlative primary tumors to those in FIG. 4 that derive from the same tissue type (kidney, breast, lung, and ovarian) and analyzed in parallel by the very same technology and under the very same set of assay conditions.

TABLE 1
Protein-Common Nonmetastatic Only

| | SwissProt# |
|---|---|
| Breast carcinoma amplified sequence 1 | O75363 |
| (Amplified and overexpressed in breast cancer) | |
| Cadherin-related tumor suppressor homolog precursor | Q14517 |
| Caspase-9 precursor (CASP-9) (Apoptotic protease Mch-6) | P55211 |
| Exostosin-like 3 (Putative tumor suppressor protein | O43909 |
| EXTL3) | |
| Fatty acid-binding protein, brain | O15540 |
| (Mammary derived growth inhibitor related) | |
| Intestinal membrane A4 protein | Q04941 |
| (Differentiation-dependent protein A4) | |
| Leucine zipper putative tumor suppressor 1 | Q9Y250 |
| Maspin precursor (Protease inhibitor 5) | P36952 |
| Rho-related GTP-binding protein RhoC | P08134 |

TABLE 2
Protein-Common Metastatic Only

| | SwissProt# |
|---|---|
| ADAM 17 precursor | P78536 |
| ADAM 7 precursor | Q9H2U9 |
| ADAMTS-5 precursor | Q9UNA0 |
| ADAMTS-5 precursor | Q9H324 |
| ADAMTS-16 precursor | Q8TE57 |
| ADAMTS-9 precursor | Q9P2N4 |
| Carcinoembryonic antigen-related cell adhesion molecule 5 precursor | P06731 |
| Carcinoembryonic antigen-related cell adhesion molecule 6 precursor | P40199 |
| Epithelial-cadherin precursor (E-cadherin) | P12830 |
| Extracellular matrix protein FRAS1 precursor | Q86XX4 |
| Integrin alpha-E precursor (Mucosal lymphocyte-1 antigen) | P38570 |
| Integrin beta-1 precursor (Fibronectin receptor beta subunit) | P05556 |
| Integrin beta-8 precursor | P26012 |
| Integrin-linked protein kinase 2 (ILK-2) | P57043 |
| Inter-alpha-trypsin inhibitor heavy chain H3 precursor | Q06033 |
| Lamina-associated polypeptide 2 isoform alpha | P42166 |
| Lamina-associated polypeptide 2, isoforms beta/gamma | P42167 |
| Laminin alpha-2 chain precursor (Laminin M chain) (Merosin heavy chain) | P24043 |
| Laminin alpha-3 chain precursor (Epiligrin 170 kDa subunit) | Q16787 |
| Laminin beta-2 chain precursor (S-laminin) | P55268 |
| Laminin beta-3 chain precursor (Laminin 5 beta 3) | Q13751 |
| Ligatin (Hepatocellular carcinoma-associated antigen 56) | P41214 |
| LYRIC protein (Metastasis adhesion protein) | Q86UE4 |
| Matrix extracellular phosphoglycoprotein precursor | Q9NQ76 |
| Mesothelin precursor (CAK1 antigen) | Q13421 |
| Mucin 1 precursor (MUC-1) | P15941 |
| Mucin 2 precursor (Intestinal mucin 2) | Q02817 |
| Nucleoside diphosphate kinase A (Tumor metastatic process-associated protein) | P15531 |
| Pleckstrin homology domain containing family C member 1 | Q96AC1 |
| Pleckstrin homology domain-containing protein family B member 2 | Q96CS7 |
| Protocadherin 15 precursor | Q96QU1 |
| Protocadherin 16 precursor (Cadherin-19) | Q96JQ0 |
| Protocadherin 9 precursor | Q9HC56 |
| Protocadherin alpha 2 precursor (PCDH-alpha2) | Q9Y5H9 |
| Protocadherin alpha C1 precursor (PCDH-alpha-C1) | Q9H158 |
| Protocadherin beta 9 precursor (PCDH-beta9) | Q9Y5E1 |
| Protocadherin Fat 2 precursor (hFat2) | Q9NYQ8 |
| Protocadherin gamma A7 precursor (PCDH-gamma-A7) | Q9Y5G6 |
| Putative transmembrane protein NMB precursor | Q14956 |
| Stromelysin-2 precursor (Matrix metalloproteinase-10) | P09238 |

TABLE 3A
Protein in Primary Ovary Cancer #1 - metastasis positive

| | SwissProt# |
|---|---|
| Drebrin-like protein (Cervical mucin-associated protein) | Q9UJU6 |
| ADAM 17 precursor (A disintegrin and metalloproteinase | P78536 |
| domain 17) | |
| Tenascin precursor(Glioma-associated-extracellular matrix | P24821 |
| antigen) | |
| Alpha-1 catenin (Cadherin-associated protein) | P35221 |
| Cadherin EGF LAG seven-pass G-type receptor 3 precursor | Q9NYQ7 |
| Catenin delta-1 (p120 catenin)Cadherin-associated Src | O60716 |
| substrate) | |
| Epithelial-cadherin precursor (E-cadherin) | P12830 |
| Protocadherin 16 precursor(Cadherin-19) | Q96JQ0 |
| Protocadherin 9 precursor | Q9HC56 |
| Protocadherin alpha 2 precursor (PCDH-alpha2) | Q9Y5H9 |
| Protocadherin beta 9 precursor (PCDH-beta9) | Q9Y5E1 |
| Integrin beta-4 precursor (GP150) (CD104 antigen) | P16144 |

TABLE 3B
Proteins in Primary Ovary Cancer #2 - metastasis positive

| | SwissProt# |
|---|---|
| Extracellular matrix protein FRAS1 precursor | Q86XX4 |
| Integrin alpha-E precursor (Mucosal lymphocyte-1 antigen) | P38570 |
| Protocadherin 15 precursor | Q96QU1 |
| Alpha-1 catenin (Cadherin-associated protein) | P35221 |
| Epithelial-cadherin precursor (E-cadherin) | P12830 |

TABLE 3C
Protein in Primary Kidney Cancer - metastasis positive

| | SwissProt# |
|---|---|
| ADAMTS-10 precursor (A disintegrin and | Q9H324 |
| metalloproteinase with thrombospondin motifs 10) | |
| Tenascin precursor (Glioma-associated-extracellular | P24821 |
| matrix antigen) | |
| Alpha-1 catenin (Cadherin-associated protein) | P35221 |
| Cadherin-13 precursor | P55290 |
| Protocadherin 15 precursor | Q96QU1 |
| Protocadherin 16 precursor | Q96JQ0 |

TABLE 3D
Proteins in Primary Lung Cancer #1 - metastasis positive

| | SwissProt# |
|---|---|
| ADAMTS-16 precursor (A disintegrin and metalloproteinase with thrombospondin motifs 16) | Q8TE57 |
| Alpha-1 catenin (Cadherin-associated protein) (Alpha E-catenin) | P35221 |
| Tenascin precursor (Glioma-associated-extracellular matrix antigen) | P24821 |
| Carcinoembryonic antigen-related cell adhesion molecule 6 precursor | P40199 |
| Matrix extracellular phosphoglycoprotein precursor | Q9NQ76 |

TABLE 3E
Proteins in Primary Breast Cancer #1 - metastasis positive

| | SwissProt# |
|---|---|
| Drebrin-like protein (Cervical mucin-associated protein) | Q9UJU6 |
| Mucin 1 precursor (Tumor-associated epithelial membrane | P15941 |
| antigen) | |
| Tenascin precursor (Glioma-associated-extracellular matrix | P24821 |
| antigen) | |
| Alpha-1 catenin (Cadherin-associated protein) | P35221 |
| Protocadherin Fat 2 precursor | Q9NYQ8 |

TABLE 3F
Proteins in Primary Breast Cancer #2 - metastasis positive

| | SwissProt# |
|---|---|
| Alpha-1 catenin (Cadherin-associated protein) | P35221 |
| Catenin delta-1 | O60716 |
| Protocadherin gamma A7 precursor | Q9Y5G6 |
| ADAM 7 precursor (A disintegrin and metalloproteinase | Q9H2U9 |
| domain 7) | |
| Tenascin precursor (Glioma-associated-extracellular | P24821 |
| matrix antigen) | |
| Stromelysin-2 precursor (Matrix metalloproteinase-10) | P09238 |
| ADAMTS-5 precursor (A disintegrin and metalloproteinase | Q9UNA0 |
| with thrombospondin motifs 5) | |

TABLE 4A
Proteins in Primary Breast Cancer - metastasis negative

| | SwissProt# |
|---|---|
| Basement membrane-specific heparan sulfate proteoglycan core protein (HSPG) | P98160 |
| Cadherin-23 precursor (Otocadherin) | Q9H251 |
| DJ-1 protein (Oncogene DJ1) | Q99497 |
| Dual specificity protein phosphatase (CDC14) | O60729 |
| Exostosin-like 3 (Putative tumor suppressor protein EXTL3) | O43909 |
| Galectin-3 binding protein precursor (Tumor-associated antigen 90K) | Q08380 |
| Importin-alpha re-exporter (Cellular apoptosis susceptibility protein) | P55060 |
| Ras GTPase-activating-like protein IQGAP1 (p195) | P46940 |
| Ras-related protein Rab-1B | Q9H0U4 |
| Ras-related protein Rab-7 | P51149 |
| Ras-related protein Rap-1b | P61224 |
| Regulator of G-protein signaling 17 (RGS17) | Q9UGC6 |
| Src substrate cortactin (Oncogene EMS1) | Q14247 |
| Tyrosine-protein phosphatase, non-receptor type 1 | P18031 |
| Vascular endothelial growth factor receptor 1 precursor (VEGFR-1) | P17948 |

TABLE 4B
Proteins in Primary Kidney Cancer - metastasis negative

| | SwissProt# |
|---|---|
| Alpha-1 catenin (Cadherin-associated protein) (Alpha E-catenin) | P35221 |
| Apoptosis regulator BAX | Q07814 |
| Basement membrane-specific heparan sulfate proteoglycan core protein precursor (HSPG) | P98160 |
| Basigin precursor (CD147 antigen) (Tumor cell-derived collagenase stimulatory factor) | P35613 |
| Carbonic anhydrase IX precursor (Renal cell carcinoma-associated antigen) | Q16790 |
| DJ-1 protein (Oncogene DJ1) | Q99497 |
| EH-domain containing protein 4 (Hepatocellular carcinoma-associated protein) | Q9H223 |
| Endoplasmin precursor (94 kDa glucose-regulated protein) (Tumor rejection antigen 1) | P14625 |
| Fatty acid-binding protein, brain (B-FABP)(Mammary derived growth inhibitor related) | O15540 |
| Fatty acid-binding protein, heart (H-FABP) (Mammary-derived growth inhibitor) | P05413 |
| Fructose-bisphosphate aldolase A (Lung cancer antigen NY-LU-1) | P04075 |
| Galectin-3 binding protein precursor (Tumor-associated antigen 90K) | Q08380 |
| Hepatoma-derived growth factor (HDGF) | P51858 |
| Heterogeneous nuclear ribonucleoprotein K (Transformation up-regulated nuclear protein) | P61978 |
| Leucine zipper putative tumor suppressor 1 | Q9Y250 |
| Phosphatidylethanolamine-binding protein (PEBP) (Prostatic binding protein) | P30086 |
| Programmed cell death protein 5 (TF-1 cell apoptosis related gene-19 protein) | O14737 |
| Programmed cell death protein 8 (Apoptosis-inducing factor) | O95831 |
| Proliferation-associated protein 2G4 (Cell cycle protein p38-2G4 homolog) | Q9UQ80 |
| Ras and Rab interactor 1 (Ras interaction/interference protein 1) | O13671 |
| Ras GTPase-activating-like protein IQGAP1 | P46940 |
| Ras-related protein R-Ras (p23) | P10301 |
| Ras-related protein Rab-1A (YPT1-related protein) | P62820 |
| Ras-related protein Rab-2B | Q8WUD1 |
| Ras-related protein Rab-5C | P51148 |
| Ras-related protein Rab-7 | P51149 |
| Ras-related protein Rap-1b | P61224 |
| TATA-binding protein associated factor 2N (Oncogene FUS) | Q92804 |
| Tenascin precursor (TN) (Glioma-associated-extracellular matrix antigen) | P24821 |
| Tumor protein D52 (N8 protein) | P55327 |
| Tumor protein D54 (hD54) (D52-like 2) | O43399 |

TABLE 4C
Proteins in Primary Kidney Cancer - metastasis negative

| | SwissProt# |
|---|---|
| Alpha-1 catenin (Cadherin-associated protein) (Alpha E-catenin) | P35221 |
| Apoptosis regulator BAX | Q07814 |
| Basement membrane-specific heparan sulfate proteoglycan core protein precursor (HSPG) | P98160 |
| Basigin precursor (CD147 antigen) (Tumor cell-derived collagenase stimulatory factor) | P35613 |
| Carbonic anhydrase IX precursor (Renal cell carcinoma-associated antigen) | Q16790 |
| DJ-1 protein (Oncogene DJ1) | Q99497 |
| EH-domain containing protein 4 (Hepatocellular carcinoma-associated protein) | Q9H223 |
| Endoplasmin precursor (94 kDa glucose-regulated protein) (Tumor rejection antigen 1) | P14625 |
| Fatty acid-binding protein, brain (B-FABP)(Mammary derived growth inhibitor related) | O15540 |
| Fatty acid-binding protein, heart (H-FABP) (Mammary-derived growth inhibitor) | P05413 |
| Fructose-bisphosphate aldolase A (Lung cancer antigen NY-LU-1) | P04075 |
| Galectin-3 binding protein precursor (Tumor-associated antigen 90K) | Q08380 |
| Hepatoma-derived growth factor (HDGF) | P51858 |
| Heterogeneous nuclear ribonucleoprotein K (Transformation up-regulated nuclear protein) | P61978 |
| Leucine zipper putative tumor suppressor 1 | Q9Y250 |
| Phosphatidylethanolamine-binding protein (PEBP) (Prostatic binding protein) | P30086 |
| Programmed cell death protein 5 (TF-1 cell apoptosis related gene-19 protein) | O14737 |
| Programmed cell death protein 8 (Apoptosis-inducing factor) | O95831 |
| Proliferation-associated protein 2G4 (Cell cycle protein p38-2G4 homolog) | Q9UQ80 |
| Ras and Rab interactor 1 (Ras interaction/interference protein 1) | Q13671 |
| Ras GTPase-activating-like protein IQGAP1 | P46940 |
| Ras-related protein R-Ras (p23) | P10301 |
| Ras-related protein Rab-1A (YPT1-related protein) | P62820 |
| Ras-related protein Rab-2B | Q8WUD1 |
| Ras-related protein Rab-5C | P51148 |
| Ras-related protein Rab-7 | P51149 |
| Ras-related protein Rap-1b | P61224 |
| TATA-binding protein associated factor 2N (Oncogene FUS) | Q92804 |
| Tenascin precursor (TN) (Glioma-associated-extracellular matrix antigen) | P24821 |
| Tumor protein D52 (N8 protein) | P55327 |
| Tumor protein D54 (hD54) (D52-like 2) | O43399 |

TABLE 4D
Proteins in Primary Lung Cancer - metastasis negative

| | SwissProt# |
|---|---|
| 60S ribosomal protein L10 (QM protein) (Tumor suppressor QM) | P27635 |
| 60S ribosomal protein L6 (Neoplasm-related protein C140) | Q02878 |
| Alpha-1 catenin (Cadherin-associated protein) (Alpha E-catenin) | P35221 |
| Basement membrane-specific heparan sulfate proteoglycan core protein precursor (HSPG) | P98160 |
| Carbonic anhydrase IX precursor (Renal cell carcinoma-associated antigen G250) | Q16790 |
| Defender against cell death 1 (DAD-1) | P61803 |
| Endoplasmin precursor (94 kDa glucose-regulated protein) (Tumor rejection antigen 1) | P14625 |
| Fructose-bisphosphate aldolase A (Lung cancer antigen NY-LU-1) | P04075 |
| Galectin-3 binding protein precursor (Tumor-associated antigen 90K) | Q08380 |
| Hepatoma-derived growth factor (HDGF) | P51858 |
| Importin-alpha re-exporter (Cellular apoptosis susceptibility protein) | P55060 |
| Mitogen-activated protein kinase 1 (Extracellular signal-regulated kinase 2) (ERK-2) | P28482 |
| Ras GTPase-activating-like protein IQGAP1 | P46940 |
| Ras-related protein Rab-10 | P61026 |
| Ras-related protein Rab-14 | P61106 |
| Ras-related protein Rab-1A (YPT1-related protein) | P62820 |
| Ras-related protein Rab-5C (RAB5L) (L1880) | P51148 |
| Ras-related protein Rab-7 | P51149 |
| Serine/threonine protein phosphatase 2A (Medium tumor antigen-associated protein) | P30153 |
| Stathmin (Phosphoprotein p19) (Oncoprotein 18) | P16949 |
| TATA-binding protein associated factor 2N (Oncogene FUS) | Q92804 |
| Tenascin precursor (TN) (Glioma-associated-extracellular matrix antigen) | P24821 |
| Tumor protein D52 (N8 protein) | P55327 |
| Tumor protein D54 (hD54) (D52-like 2) | O43399 |

What is claimed is:

1. A method of assessing a metastatic status of a test primary tumor, comprising the steps of:
(a) identifying one or more test proteins expressed by the test primary tumor to create a test protein profile;
(b) comparing the test protein profile with a reference protein profile, wherein the reference protein profile comprises one or more reference proteins and is obtained from modified samples of one or more reference primary tumors that are known to have become metastatic, wherein the presence of one or more proteins in the test protein profile and the reference protein profile indicates the metastatic status of the test primary tumor.

2. The method of claim 1, wherein the modified samples of one or more primary tumors comprise histopathologically processed fresh tissue or frozen tissue.

3. The method of claim 2, wherein the histopathologically processed fresh tissue or frozen tissue is contacted with a reaction buffer and heated at a temperature and for a time sufficient to reduce protein cross-linking in the tissue, and then treated with an effective amount of a proteolytic enzyme for a time sufficient to disrupt tissue and cellular structure of the tissue.

4. The method of claim 2, wherein the histopathologically processed frozen or fresh tissue is selected from the group consisting of formalin-fixed tissue, formalin-fixed cells, formalin-fixed and paraffin embedded (FFPE) tissue, FFPE cells, FFPE tissue blocks, FFPE cells from primary tumors, FFPE tissue blocks from biopsies obtained surgically from primary tumors, and formalin fixed or paraffin embedded tissue culture cells derived from a primary tumor.

5. The method of claim 1, wherein the test protein profile and the reference protein profile each contain two or more proteins.

6. The method of claim 1, wherein the reference protein profile comprises a protein selected from the group of proteins listed in Tables 1-4.

7. The method of claim 1, wherein the reference protein profile comprises three proteins selected from the group of proteins listed in Tables 1-4.

8. The method of claim 1, wherein the reference protein profile comprises five proteins selected from the group of proteins listed in Tables 1-4.

9. The method of claim 1, wherein the modified samples of one or more reference primary tumors comprise protein samples prepared using Liquid Tissue® reagents and protocols.

10. The method of claim 1, wherein the reference protein profile is obtained from modified reference samples of two or more primary tumors.

11. The method of claim 1, wherein the test primary tumor is of the same type of tumor as the reference primary tumor.

12. The method of claim 1, wherein the test primary tumor is an ovarian, kidney, lung or breast tumor.

13. The method of claim 1, wherein the reference protein profile comprises a matrix metalloprotease, a metalloprotease, a cadherin, a protocadherin, an integrin, an extracellular matrix protein, an enzyme that digests extracellular matrix, a motility protein, a cell adhesion protein, a membrane invasion protein, a membrane disintegration protein, a basement membrane disintegration protein, a basement membrane invasion protein, or a cell motility protein.

14. The method of claim 1, wherein the one or more test proteins are identified by mass spectroscopy.

15. The method of claim 1, wherein the one or more reference proteins are identified by a mass spectroscopy technique.

16. The method of claim 16, wherein the mass spectrometry technique is selected from the group consisting of LC-ESI-MS, LC-ESI-MS/MS, LC-nanospray-MS, LC-nanospray-MS/MS, MALDI-TOF, MALDI-TOF/TOF, SELDI-TOF, and SELDI-TOF/TOF.

17. The method of claim 1, wherein the identification of one or more test proteins is performed using a protein array or an immuno-based assay.

18. A method of assessing a metastatic status of a test primary tumor, comprising the steps of:
(a) determining the amount one or more test proteins expressed by the test primary tumor to create a scaled test protein profile;
(b) comparing the test protein profile with a reference protein profile, wherein the reference protein profile contains the relative amounts of one or more reference proteins and is obtained from modified samples of one or more reference primary tumors that are known to have become metastatic, wherein the presence of a protein in the scaled test protein profile at a level about equal to or greater than the amount of the protein in the reference protein profile indicates the metastatic status of the test primary tumor.

19. A method for generating a metastatic tumor protein profile, comprising:
a) collecting a first sample comprising material obtained from a first primary tumor that is known to have metastasized in a subject, wherein the material contains at least one protein expressed in a tumor cell of the first primary tumor;
b) collecting a second sample comprising material obtained from a second primary tumor that is known to have not metastasized in a subject, wherein the material contains at least one protein expressed in a tumor cell of the second primary tumor;
c) separately modifying the first sample and the second sample, comprising the steps of:
i) contacting the material with a reaction buffer and heating the contacted material at a temperature and for a time sufficient to negatively affect protein cross-linking in the tissue, and
ii) treating the heated material with an effective amount of a proteolytic enzyme for a time sufficient to disrupt the tissue and cellular structure of the tissue;
d) determining the amount one or more proteins in the modified first and second samples; and,
e) identifying proteins that are:
i) detectable in the first sample but not the second sample, and
ii) detectable in higher amounts in the first sample than in the second sample,
thereby generating a metastatic tumor protein profile.

20. A kit comprising in one or more containers two or more proteins selected from the group consisting of the proteins listed in Tables 1-4, or antibodies that are immunospecific therefor.

21. The kit of claim 20, comprising five or more proteins selected from the group consisting of the proteins listed in Tables 1-4, or antibodies that are immunospecific therefor.
